(19) United States
(12) Patent Application Publication
(10) Pub. No.: US 2015/0283148 A1
(43) Pub. Date: Oct. 8, 2015

(54) ARRANGEMENT OF SPECIFIC ANTIDOTES WITH ALLOPATHIC MEDICINAL PREPARATIONS IN GENERAL SECONDARY PACKAGE

(71) Applicant: Anatoly Zazulia, Krasnodar (RU)

(72) Inventor: Anatoly Zazulia, Krasnodar (RU)

(21) Appl. No.: 14/360,739

(22) PCT Filed: Apr. 8, 2014
(86) PCT No.: PCT/RU2014/000258

§ 371 Date: May 27, 2014

(51) Int. Cl. A61K 31/5415; A61K 45/06; A61K 31/4515

(52) U.S. Cl. CPC A61K 31/5415; A61K 31/4515; A61K 45/06

(57) ABSTRACT

The invention relates to the area of medicine, namely to the medicinal preparations, applied in clinical practice. The proposed medicinal preparation contains two non-miscible components, one allopathic medicinal preparation, and the second one is a homeopathic medicinal preparation. It features the second component obtained by potentiating of the first component diluted within the range from 6C to 30C and placed into blisters, con bottles, ampoules in the general secondary package with an allopathic component. Upon the termination or cancellation of an allopathic treatment course, the use of specific antidote—homeopathic component corrects the side effects, reduces the allergic reactions, accelerates the restorative processes in organism.

The invention relates to the region of medicine, namely to the medicinal preparations for application in medical practice. It is an object of invention to correct both the latent side effects and those already known from the practice of the allopathic medicinal preparations through the use of specific antidotes, which are in the same secondary package with an allopathic medicinal preparation.

It is common knowledge the method of a human organism treatment by a complex medicinal preparation, containing at least, a single allopathic medicinal preparation, and additionally containing, at least, a single homeopathic medicinal preparation diluted from 3D to 12D. (Patent RU2136305, Classes A61K 35/78, A61K 31/00, A61K 33/00, 1999.) The drawback of the known method of treatment is that the balanced selection of the components results in the stronger effect of allopathic medicinal preparation caused by homeopathic one, in other words, the side effects and contraindications of an allopathic preparation may become stronger and increase. The drawback of the known patent is failing description of regularities of mixing the components, through which the natural protective functions become more active, and the restoration “programs” are set for an organism.

There are known facilities for enhancement of remedies that follow from the state of the art. (Patent RU2253478C1 class A61K45/00, 2003). The drawback, how the patent's author found out, is the non-summable enhancement of remedy's medical. effects and possibility of appearance of side effects limit a clinical use of present decision. The drawback of known method is that activated form of minute dose of remedy place into the therapeutic dose of the same remedy. It provokes multidirectional biochemical reactions in organism, and in the issue it will be difficult to choose the therapeutic dose for patients cure. This patent has no definitions of small and oversmall doses, it is no clear what does the author means by these definitions. The bounds of dilutions are not mentioned in the patent claim, abstract and description of the invention. The drawback of that method is that the author doesn't extrapolate the dilutions, which were mentioned in examples of a patent, to the other remedies, There are no instructions, what kinds of potencies are necessary for other medicinal preparations or substances of groups of medicinal preparations in the anatomic-therapeutic-chemical classification system (WHO, 2000).

There are known homeopathic medicinal preparations (RU 21.22858 C1, A 61 K 35/78 1998; RU 2133123 C1, A 61 K 35/78, 1999; RU2177795 C1, A 61 K 35/78, 2002; RU 2182492 C1, A 61 K 39/00, 2002; RU 2191601 C1, A 61 K 39/395, 2002; RU 2192882 C1, A 61 K 38/22, 2002; RU 2201255 C1, A 61 K 39/395, 2703.2003; RU 2209083 C1, A 61 K 39/395, 27, Jul. 2003), These preparations are available for individualization of cure of various illnesses. it based. on similarity law.

It is known that the American bioengineers worked out a mathematical algorithm, the application of which permits to determine the side effects, caused by the different medical preparations and their joint application. The discovered regularities are the basis to determine the latent side effects for 1332 preparations. The algorithm <<invented>> 329 earlier unknown side effects on the average for the medical preparations. On the average 69 such effects were described for a single preparation. For example: the tendon tears provoked by acceptance of Norfloxacin antibiotic, the suicidal thoughts provoked by Zonisamide preparation were predicted, (<<Drug data reveal sneaky side effects>>—Nature News, 14, Mar. 2012;)

The first and primary principle of homeopathy is a “law of similarity”, which consists in that the medicinal preparations in small doses are efficient with the same diseases, provoked by these substances in the large doses. (“The homeopathic clinical pharmacology”, Ernst Farrington, Kiev, 1992, Page 3). The consequence of the “law of similarity” is it's another unexpected wording—the homeopathic preparation potentiated to the small doses is efficient with the side effects, provoked by the same allopathic preparation in therapeutic doses, in other words is its specific antidote.

The utility model is for creation of effective equipment for soft and safety treatment termination with allopathic preparations. The utility model aims at normalization of side effects and allopathic cure allergy (including oncological patients).

The solution of assigned task was arrived thanks to early unknown property that the author found out, namely to ensure the side effects normalization of allopathic cure after the termination of a course with help of homeopathic doses of the same preparation.

Further are given the concrete examples, illustrating the proposed technical solution.

### EXAMPLE 1

The homeopathic aminazine terminates the phenomena of neuroleptic syndrome, skin diseases, face and extremities edema, appearing in the process of treatment by the usual doses of aminazine in psychiatric practice.

### EXAMPLE 2

In potentiated form Stelazine 30C is also efficient as a corrector of extrapyramidal disorders, appearing in psychiatric practice with the treatment by allopathic Stelazine.

### EXAMPLE 3

The homeopathic haloperidol 30C proved itself to be a correcting agent, preventing the development of neuroleptic syndrome with the treatment by conventional allopathic haloperidol.

The foundation of invention is a phenomenon of the double-phase action of medicinal preparations, directly depending on their concentration. The majority of the medical and scientific reference books rely on this matter on Arndt-Schulz law (it is presented usually as a law), Which asserts that the low concentrations of the biological agents stimulate the physiological activity, the average concentrations suppress the physiological activity, and the high concentrations disable it totally. (Large medical encyclopedia, Arndt-Schnlz law)

An example of important clinical animal's research was published in respected magazine “The Human Toxicology”: the micro-doses of potentiated arsenic helped the rats to cleanse the organism from the material arsenic doses added to (heir food. The best results were obtained with application of 7C dilutions. The reference animals, getting the material arsenic and further getting placebo, died. (J. C. Cazin et al., <<A Study of the Effect of Decimal and Centesimal Dilution of Arsenic on Retention and Mobilization of Arsenic in the Rat>> Human Toxicology, July 1987.)

Doctor Ronald Daway, the attending, doctor of her Majesty Queen Elizabeth II, employs allopathy in his medical practice only in potentiated doses, using the side effects of the medical preparations as indications for prescription (“Discovering Homeopathy: Medicine for the 21^{st} Century” Dana Ullman).

The normalization of unknown and known side effects of allopathic preparations supports by the instrumentality of specific antidotes, which were created according to homeopathic technology. The specific antidotes are means for normalization side effects, which are created by the instrumentality of successive multiple dilution of a parent drug. And at the same time these dilutions are subjected to standardized shaking up according to homeopathic technology (Homeopathic medicinal preparations. Guidance for description and manufacture.—W. Schwabe, Moscow, p. 12-38). Meanwhile, the scale-down concentrations are made by means of serial dilution of 1 part by volume of a parent substance in 99 parts by volume (for the centesimal dilutions) of a neutral dissolvent. For each obtained dilution are performed the multiple vertical shacking up and are used separate containers for each following dilution until the receipt of necessary dose.

This homeopathic form of specific antidote is placed into the primary package and is combined with the primary package of allopathic medicinal preparation into the general secondary package. Thereafter, it delivers to the drugstores, hospitals to use it in medical practice.

Upon the termination or cancellation of an allopathic treatment course, the use of specific antidote—homeopathic component, in 6C-30C dilutions corrects the side effects, reduces the allergic reactions, and accelerates the restorative processes in organism. Thus, the proposed method permits to obtain more stable and pronounced treatment response, since a two-component nonmiscible medical preparation will treat both by allopathic indications and its side effects by specific homeopathic antidote.

Meanwhile, the homeopathic antidote of medicinal substance itself can have any significant cure effect.

The aggregate of the vast multidisciplinary modem scientific dates confirmed the biological strength of homeopathic nano-doses. (Bell, I; Koithan, M. “A model for homeopathic remedy effects: low dose nano-particles, allostatic cross-adaptation, and time-dependent sensitization in a complex adaptive System,” BMC Complementary and Alternative Medicine 2012, 12:191 doi: 10.1186/1472-6882-12-191.

Chikramane P S, Kalita D, Suresh A K, Kane S G, Bellare J R. “Why Extreme Dilutions Reach Non-zero Asymptotes: A Nanoparticulate Hypothesis Based on Froth Flotation.” Langmuir, 2012 November)

The two-component nonmiscible medicinal preparations may he represented by the pharmaceutical dosage forms as follows: tablets, powders, granules or solutions, tinctures, extracts or ointments, pastes.

The homeopathic specific tablets antidotes is a hard and. dosed pharmaceutical dosage form in homeopathy, which is produced by pressing powders and granules, containing one or several dilutions of 6C to 30C allopathic pharmaceutical substances. The basis of these preparations may be formed both by additions and the lack of addition of the special subsidiary substances or the formation of special masses. The tablets are placed into blisters and packed into general secondary package with allopathic tablets.

The homeopathic powders, granules with specific antidotes are placed into waterproof packs or plastic cans and then packed together with allopathic medicinal preparations into the single secondary package.

The sterile homeopathic solutions, tinctures, extracts with specific homeopathic antidotes are ampouled, bottled and placed into general secondary package with the liquid allopathic medicinal preparation.

The ointments, pastes with specific homeopathic antidotes are canned, tubed and packed into general secondary package with allopathic ointments and pastes.

What I claim is:

1. A medicinal preparation, comprising:
two non-miscible components, the components being placed separately into a common package, a first component is an allopathic medicinal preparation, and a second one is a homeopathic medicinal preparation being a specific antidote, wherein the second component is obtained by potentiating of the first component diluted within the range from 6C to 30C.
